(19) United States
(12) Patent Application Publication
(10) Pub. No.: US 2024/0109833 A1
(43) Pub. Date: Apr. 4, 2024

(54) METHOD FOR PREPARING GLYCOLIC ACID AND METHYL GLYCOLATE THROUGH HYDROLYSIS OF METHYL METHOXYACETATE AND METHOXYACETIC ACID

(71) Applicant: DALIAN INSTITUTE OF CHEMICAL PHYSICS, CHINESE ACADEMY OF SCIENCES, Dalian (CN)

(72) Inventors: Youming NI, Dalian (CN); Wenliang ZHU, Dalian (CN); Zhongmin LIU, Dalian (CN)

(73) Assignee: DALIAN INSTITUTE OF CHEMICAL PHYSICS, CHINESE ACADEMY OF SCIENCES, Dalian (CN)

(21) Appl. No.: 18/275,444

(22) PCT Filed: Feb. 3, 2021
(86) PCT No.: PCT/CN2021/075023

§ 371 Date: Aug. 2, 2023

(51) Int. Cl. C07C 51/377; C07C 67/31

(52) U.S. Cl. CPC C07C 51/377; C07C 67/31

(57) ABSTRACT

A method for preparing glycolic acid and methyl glycolate through hydrolysis of methyl methoxyacetate and methoxyacetic acid is provided. The method includes allowing raw materials including methyl methoxyacetate, methoxyacetic acid, and water to contact and react with a catalyst to produce glycolic acid and methyl glycolate, where the catalyst is at least one selected from the group consisting of a solid acid catalyst, a liquid acid catalyst, a solid base catalyst, and a liquid base catalyst. The method for preparing glycolic acid and methyl glycolate in the present application can be implemented by a traditional fixed-bed reactor, tank reactor, or catalytic distillation reactor under an atmospheric pressure, which is very suitable for continuous production.

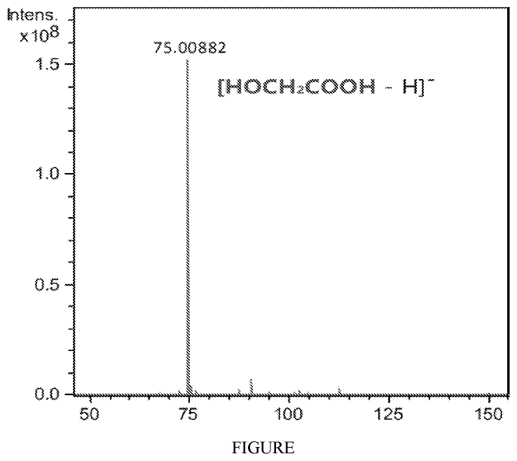

### CROSS REFERENCE TO THE RELATED APPLICATIONS

This application is the national phase entry of International Application No. PCT/CN2021/075023, filed on Feb. 3, 2021, the entire contents of which are incorporated herein by reference.

### TECHNICAL FIELD

The present application relates to a method for preparing glycolic acid and methyl glycolate through hydrolysis of methyl methoxyacetate and methoxyacetic acid, and belongs to the technical field of preparation of chemical products.

### BACKGROUND

Glycolic acid, also known as hydroxyacetic acid, is the simplest α-hydroxycarboxylic acid compound. Methyl glycolate can be hydrogenated to produce ethylene glycol (EG), and can also be easily hydrolyzed under mild conditions to produce glycolic acid. Because the molecular structure of glycolic acid includes both hydroxyl and carboxyl, glycolic acid can undergo self-polymerization to produce polyglycolic acid (PGA). PGA has not only excellent biocompatibility, but also safe biodegradability. Therefore, PGA is widely used not only for medical surgical sutures, drug sustained-release materials, and degradable human tissue scaffolds, but also in production of common plastic products. Conventional non-degradable plastic products have caused heavy environmental pollution, and biodegradable PGA plastics are expected to solve this problem. Glycolic acid can also undergo copolymerization with monomers such as lactic acid and hydroxypropionic acid to produce a polymer material with excellent performance and extensive applications. In addition, glycolic acid is an excellent chemical cleaning agent and cosmetic raw material.

Preparation methods of glycolic acid mainly include a chloroacetic acid hydrolysis method, a formaldehyde carbonylation method, and an oxalate hydrogenation/hydrolysis method. In the chloroacetic acid hydrolysis method, the preparation process of the raw material chloroacetic acid causes heavy pollution, and the hydrolysis process of chloroacetic acid leads to a large amount of waste salt, heavy pollution, and poor quality product. Thus, the chloroacetic acid hydrolysis method has been basically eliminated. The formaldehyde carbonylation method involves cheap and easily-available raw materials, but needs to be implemented under conditions such as a high temperature, a high pressure, a strong liquid acid, and an organic solvent. In addition, in the formaldehyde carbonylation method, the device is easily corroded, and the product purification is difficult, resulting in a high industrial production cost. In the oxalate hydrogenation/hydrolysis method, an oxalate is partially hydrogenated to produce methyl glycolate, and then methyl glycolate is hydrolyzed to produce glycolic acid. However, the catalyst for partial hydrogenation of the oxalate is not mature and has low conversion efficiency and poor stability; and the oxalate production process is cumbersome and costly. These problems seriously restrict the development of the oxalate hydrogenation/hydrolysis method.

In recent years, a dimethoxymethane (DMM) carbonylation reaction to prepare methyl methoxyacetate has received widespread attention. This reaction is based on a molecular sieve catalyst, can be conducted at a low reaction temperature, and has high atomic economy. The raw material DMM can be produced by a very mature industrialized technology with high efficiency, and thus is cheap.

However, in the prior art, methyl methoxyacetate produced from DMM carbonylation has not been applied to preparation of glycolic acid and methyl glycolate. In addition, methoxyacetic acid resulting from hydrolysis of an ester bond of methyl methoxyacetate is relatively less used, resulting in waste of the raw material.

### SUMMARY

According to an aspect of the present application, a method for preparing glycolic acid and methyl glycolate through hydrolysis of methyl methoxyacetate and methoxyacetic acid is provided. The method of the present application is particularly suitable for carbonylation of DMM produced in the coal chemical industry to produce methyl methoxyacetate and then hydrolysis to produce glycolic acid and methyl glycolate. This reaction process can be used in combination with DMM carbonylation for preparing methyl methoxyacetate to allow efficient, environmentally-friendly, and economical conversion of DMM (a platform chemical in the coal chemical industry) into glycolic acid and methyl glycolate, which expands the use of methyl methoxyacetate and methoxyacetic acid and brings beneficial effects.

A method for preparing glycolic acid and methyl glycolate through hydrolysis of methyl methoxyacetate and methoxyacetic acid is provided, including: allowing raw materials including the methyl methoxyacetate, the methoxyacetic acid, and water to contact and react with a catalyst to produce the glycolic acid and the methyl glycolate, where the catalyst is any one selected from the group consisting of a solid acid catalyst, a liquid acid catalyst, a solid base catalyst, and a liquid base catalyst.

In recent years, the DMM carbonylation reaction to prepare methyl methoxyacetate has received widespread attention. This reaction is based on a molecular sieve catalyst, can be conducted at a low reaction temperature, and has high atomic economy. The raw material DMM can be produced by a very mature industrialized technology with high efficiency, and thus is cheap. In the present application, glycolic acid, methyl glycolate, and methoxyacetic acid can be obtained through hydrolysis of ether and ester bonds in the methyl methoxyacetate. Because methoxyacetic acid is relatively less used and can be returned to a reactor to undergo co-hydrolysis with methyl methoxyacetate, the method of the present application becomes an environmentally-friendly and economical production path for glycolic acid and methyl glycolate.

Optionally, the solid acid catalyst is at least one selected from the group consisting of an acidic molecular sieve catalyst, an acidic resin catalyst, and an acidic alumina catalyst; and
the acidic molecular sieve catalyst includes an acidic molecular sieve.

Optionally, the acidic molecular sieve is at least one selected from the group consisting of an acidic MFI-structured molecular sieve, an acidic FAU-structured molecular sieve, an acidic FER-structured molecular sieve, an acidic BEA-structured molecular sieve, an acidic MOR-structured molecular sieve, and an acidic MWW-structured molecular sieve.

Preferably, the acidic molecular sieve is any one selected from the group consisting of an acidic MFI-structured molecular sieve and an acidic FER-structured molecular sieve.

Optionally, the acidic molecular sieve is at least one selected from the group consisting of an acidic ZSM-5 molecular sieve, an acidic Y molecular sieve, an acidic ZSM-35 molecular sieve, an acidic β molecular sieve, an acidic MOR molecular sieve, and an acidic MCM-22 molecular sieve.

Preferably, the acidic molecular sieve is at least one selected from the group consisting of an acidic ZSM-5 molecular sieve and an acidic ZSM-35 molecular sieve.

Optionally, the acidic molecular sieve is at least one selected from the group consisting of a hydrogen-type ZSM-5 molecular sieve, a hydrogen-type Y molecular sieve, a hydrogen-type ZSM-35 molecular sieve, a hydrogen-type β molecular sieve, a hydrogen-type MOR molecular sieve, and a hydrogen-type MCM-22 molecular sieve.

Preferably, the acidic molecular sieve is at least one selected from the group consisting of a hydrogen-type ZSM-5 molecular sieve and a hydrogen-type ZSM-35 molecular sieve.

Optionally, a Si/Al atom ratio of the acidic molecular sieve is 3 to 500.

Specifically, an upper limit of the Si/Al atom ratio of the acidic molecular sieve is selected from the group consisting of 20, 10, 50, 100, and 500; and a lower limit of the Si/Al atom ratio of the acidic molecular sieve is selected from the group consisting of 3, 10, 20, 50, and 100.

Preferably, a Si/Al atom ratio of the acidic molecular sieve is 20 to 500.

Optionally, a content of the acidic molecular sieve in the acidic molecular sieve catalyst is 50 wt % to 100 wt %.

Optionally, the acidic molecular sieve catalyst further includes a forming agent; the forming agent is an oxide; and the oxide is one selected from the group consisting of alumina and silicon oxide.

Optionally, a content of the forming agent in the acidic molecular sieve catalyst is m, and 0<m≤50 wt %.

Optionally, the acidic molecular sieve catalyst is a fresh acidic molecular sieve catalyst and/or a regenerated acidic molecular sieve catalyst; and the fresh acidic molecular sieve catalyst is an unused acidic molecular sieve catalyst.

Optionally, a regeneration method of the acidic molecular sieve catalyst includes: treating an inactivated acidic molecular sieve catalyst with an oxygen-containing regeneration gas at 400° C. to 800° C. for 0.5 h to 24 h to obtain the regenerated acidic molecular sieve catalyst,
where a volume fraction of oxygen in the oxygen-containing regeneration gas is 0.5% to 50%.

Optionally, the acidic resin catalyst is any strongly-acidic cation exchange resin.

Optionally, a skeleton structure in the strongly-acidic cation exchange resin is a copolymer of styrene and divinylbenzene (DVB); and
an acidic group in the strongly-acidic cation exchange resin is a sulfonic acid group.

Optionally, the acidic alumina catalyst is γ-alumina.

Optionally, the liquid acid catalyst is any acidic liquid.

Optionally, the liquid acid catalyst is at least one selected from the group consisting of sulfuric acid, hydrochloric acid, nitric acid, and phosphoric acid.

Optionally, a concentration of H⁺ in the liquid acid catalyst is 0.01 mol/L to 10 mol/L.

Optionally, the solid base catalyst is at least one selected from the group consisting of hydrotalcite, an anion exchange resin, and hydroxyapatite.

Optionally, the liquid base catalyst is any alkaline liquid.

Optionally, the liquid base catalyst is any one selected from the group consisting of a sodium hydroxide aqueous solution, a potassium hydroxide aqueous solution, a calcium hydroxide aqueous solution, and a magnesium hydroxide aqueous solution.

Optionally, a concentration of OH⁻ in the liquid base catalyst is 0.01 mol/L to 10 mol/L.

Optionally, conditions of the reaction are as follows:
a reaction temperature is 60° C. to 260° C.;
a reaction pressure is 0.1 MPa to 10 MPa; and
in the raw materials, a ratio of a total mole number of the methyl methoxyacetate and the methoxyacetic acid to a mole number of the water is 1:2 to 1:20 and
a ratio relationship between the methyl methoxyacetate and the methoxyacetic acid is not limited.

Specifically, an upper limit of the reaction temperature is selected from the group consisting of 130° C., 160° C., 200° C., and 260° C.; and a lower limit of the reaction temperature is selected from the group consisting of 60° C., 130° C., 160° C., and 200° C.

An upper limit of the reaction pressure is selected from the group consisting of 0.3 MPa, 1 MPa, 5 MPa, and 10 MPa; and a lower limit of the reaction pressure is selected from the group consisting of 0.1 MPa, 0.3 MPa, 1 MPa, and 5 MPa.

An upper limit of the ratio of a total mole number of the methyl methoxyacetate and the methoxyacetic acid to a mole number of the water is selected from the group consisting of 1:3, 1:6, 1:8, 1:10, 1:15, and 1:20; and a lower limit of the ratio of a total mole number of the methyl methoxyacetate and the methoxyacetic acid to a mole number of the water is selected from the group consisting of 1:2, 1:3, 1:6, 1:8, 1:10, and 1:15.

Preferably, the conditions of the reaction are as follows:
the reaction temperature is 130° C. to 200° C.;
the reaction pressure is 0.1 MPa to 0.3 MPa; and
in the raw materials, a ratio of a total mole number of the methyl methoxyacetate and the methoxyacetic acid to a mole number of the water is 1:3 to 1:8 and
a molar ratio of the methyl methoxyacetate to the methoxyacetic acid is 4:1 to 9:1.

Specifically, an upper limit of the molar ratio of the methyl methoxyacetate to the methoxyacetic acid is selected from the group consisting of 5:1 and 9:1; and a lower limit of the molar ratio of the methyl methoxyacetate to the methoxyacetic acid is selected from the group consisting of 4:1 and 5:1.

Optionally, the reaction is conducted in a reactor; and
the reactor is any one selected from the group consisting of a fixed-bed reactor, a tank reactor, and a catalytic distillation reactor.

Optionally, the reactor includes a single fixed-bed reactor, or a plurality of fixed-bed reactors connected in series and/or parallel; or
the reactor includes a single tank reactor, or a plurality of tank reactors connected in series and/or parallel; or
the reactor includes a single catalytic distillation reactor, or a plurality of catalytic distillation reactors connected in series and/or parallel.

Optionally, when the fixed-bed reactor is adopted, a weight hourly space velocity (WHSV) of the methyl methoxyacetate and the methoxyacetic acid in the raw materials is 0.1 h⁻¹ to 3 h⁻¹.

Specifically, an upper limit of the WHSV of the methyl methoxyacetate and the methoxyacetic acid is selected from the group consisting of 0.6 h⁻¹, 1 h⁻¹, and 3 h⁻¹; and a lower limit of the WHSV of the methyl methoxyacetate and the methoxyacetic acid is selected from the group consisting of 0.1 h⁻¹, 0.6 h⁻¹, and 1 h⁻¹.

Optionally, when the tank reactor is adopted, a stirring speed is 250 rpm to 350 rpm; and a reaction time is 1 d to 3 d.

Optionally, when the catalytic distillation reactor is adopted, a reaction time is 8 h to 15 h; a stirring speed is 350 rpm to 650 rpm; and a reflux ratio is 1 to 3.

Optionally, the methyl methoxyacetate in the raw materials includes a freshly-added raw material and/or unreacted methyl methoxyacetate left after a product is separated; and/or, the methoxyacetic acid in the raw materials includes a freshly-added raw material and/or unreacted methoxyacetic acid left after a product is separated; and/or, the water in the raw materials includes a freshly-added raw material and/or unreacted water left after a product is separated.

Specifically, in an embodiment, the methyl methoxyacetate, methoxyacetic acid, and water in the raw materials each are a freshly-added raw material and/or an unreacted material left after a product is separated.

Optionally, the reaction is conducted in an inactive atmosphere; and
the inactive atmosphere includes any one selected from the group consisting of nitrogen and an inert gas.

Possible beneficial effects of the present application:
1) The method for preparing glycolic acid and methyl glycolate in the present application can be implemented by a traditional fixed-bed reactor, tank reactor, or catalytic distillation reactor under an atmospheric pressure, which is very suitable for continuous production.
2) When used in combination with methanol and formaldehyde condensation to prepare DMM and DMM carbonylation to prepare methyl methoxyacetate, the method in the present application can allow efficient, environmentally-friendly, and economical conversion of methanol (a platform chemical in the coal chemical industry) into glycolic acid and methyl glycolate.

### BRIEF DESCRIPTION OF THE DRAWINGS

FIGURE is a negative-ion mass spectrum of glycolic acid among reaction products analyzed by liquid chromatography-mass spectrometry in Example 1 of the present application.

### DETAILED DESCRIPTION OF THE EMBODIMENTS

The present application will be described in detail below with reference to embodiments, but the present application is not limited to these embodiments.

Possible embodiments are described below.

According to the problems faced by the existing production technologies of glycolic acid and methyl glycolate, the present application discloses a method for preparing glycolic acid and methyl glycolate through hydrolysis of methyl methoxyacetate and methoxyacetic acid. The method of the present application is particularly suitable for carbonylation of DMM produced in the coal chemical industry to produce methyl methoxyacetate and then hydrolysis to produce glycolic acid and methyl glycolate.

Specifically, the present application provides a method for preparing glycolic acid and methyl glycolate through hydrolysis of methyl methoxyacetate and methoxyacetic acid, where raw materials of methyl methoxyacetate, methoxyacetic acid, and water are allowed to pass through a reaction zone loaded with a catalyst, such that the raw materials undergo a reaction under specified conditions to produce glycolic acid and methyl glycolate.

The catalyst is one or a mixture of two or more selected from the group consisting of a solid acid catalyst, a liquid acid catalyst, a solid base catalyst, and a liquid base catalyst.

The reaction zone includes a single fixed-bed reactor, or a plurality of fixed-bed reactors connected in series and/or parallel; or a single tank reactor, or a plurality of tank reactors connected in series and/or parallel; or a single catalytic distillation reactor, or a plurality of catalytic distillation reactors connected in series and/or parallel.

Conditions of the reaction are as follows: a reaction temperature is 60° C. to 260° C.; in the raw materials, a molar ratio of methyl methoxyacetate+methoxyacetic acid to water is 1:20 to 1:2, and a molar ratio of methyl methoxyacetate to methoxyacetic acid is any ratio; and a reaction pressure is 0.1 MPa to 10 MPa.

A reaction equation for hydrolysis of methyl methoxyacetate is as follows:

CH₃OCH₂COOCH₃+2 H₂O=2CH₃OH+HOCH₂COOH (1).

There are also two partial hydrolysis reactions as follows:

CH₃OCH₂COOCH₃+H₂O═CH₃OH+HOCH₂COOCH₃ (2) and

CH₃OCH₂COOCH₃+H₂O═CH₃OH+CH₃OCH₂COOH (3).

Methoxyacetic acid in reaction (3) is further hydrolyzed under the same catalyst and reaction conditions to produce glycolic acid:

CH₃OCH₂COOH+H₂O═CH₃OH+HOCH₂COOH (4).

Reactions (1) to (4) are reversible reactions. In addition, methanol resulting from hydrolysis can also be partially dehydrated to produce dimethyl ether (DME).

The solid acid catalyst is one or a mixture of two or more selected from the group consisting of an acidic molecular sieve catalyst, an acidic resin catalyst, and an acidic alumina catalyst.

In addition to an acidic molecular sieve, the acidic molecular sieve catalyst further includes a catalyst forming agent with a weight percentage of 0% to 50%; and the catalyst forming agent is one selected from the group consisting of alumina and silicon oxide.

The acidic molecular sieve catalyst is a fresh acidic molecular sieve catalyst and/or a regenerated acidic molecular sieve catalyst.

A preparation method of the regenerated acidic molecular sieve catalyst includes: using a gas including oxygen in a volume fraction of 0.5% to 50% to treat an inactivated acidic molecular sieve catalyst obtained after hydrolysis of methyl methoxyacetate and methoxyacetic acid at 400° C. to 800° C. for 0.5 h to 24 h.

The acidic resin catalyst is a strongly-acidic cation exchange resin.

A skeleton structure in the strongly-acidic cation exchange resin is a copolymer of styrene and DVB; and an acidic group in the strongly-acidic cation exchange resin is a sulfonic acid group.

The acidic alumina catalyst is γ-alumina.

The γ-alumina is prepared by calcining an SB powder at 400° C. to 800° C.

The liquid acid catalyst is an acidic liquid.

The liquid acid catalyst is one or more selected from the group consisting of sulfuric acid, hydrochloric acid, nitric acid, and phosphoric acid.

A concentration of H⁺ in the liquid acid catalyst is 0.01 mol/L to 10 mol/L.

The solid base catalyst is one or more selected from the group consisting of hydrotalcite, an anion exchange resin, and hydroxyapatite.

A composition of the hydrotalcite can be expressed as [Mg₁₋ₓAlₓ(OH)₂]^{x+}[CO₃²⁻]_{x/2}·n H₂O, where x is 0.1 to 0.34 and n is an integer of 0 to 4; and Mg can undergo isomorphous replacement by Zn, Fe, Co, Ni, and Cu and Al can be replaced by Cr, Fe, and In.

A composition of the hydroxyapatite can be expressed as Ca₁₀₋ₓ(HPO₄)ₓ(PO₄)₆₋ₓ(OH)₂₋ₓ, where x is 0 to 1.

The liquid base catalyst is an alkaline liquid.

The liquid base catalyst is one or more selected from the group consisting of a sodium hydroxide aqueous solution, a potassium hydroxide aqueous solution, a calcium hydroxide aqueous solution, and a magnesium hydroxide aqueous solution.

A concentration of OH⁻ in the liquid base catalyst is 0.01 mol/L to 10 mol/L. Conditions of the reaction are as follows: a reaction temperature is 130° C. to 200° C.; in the raw materials, a molar ratio of methyl methoxyacetate+methoxyacetic acid to water is 1:8 to 1:3, and a molar ratio of methyl methoxyacetate to methoxyacetic acid is 4:1 to 9:1; and a reaction pressure is 0.1 MPa to 0.3 MPa.

When the reaction zone includes a single fixed-bed reactor or a plurality of fixed-bed reactors connected in series and/or parallel, a WHSV of the methyl methoxyacetate and the methoxyacetic acid in the raw materials is 0.1 h⁻¹ to 3 h⁻¹.

The methyl methoxyacetate, methoxyacetic acid, and water in the raw materials each are a freshly-added raw material and/or an unreacted raw material left after a product is separated.

When the raw materials are allowed to pass through a reaction zone loaded with an acidic molecular sieve catalyst, an inert carrier gas selected from the group consisting of nitrogen and argon is introduced.

Unless otherwise specified, the raw materials in the embodiments of the present application all are purchased from commercial sources.

Analysis methods and conversion rate and selectivity calculation in the embodiments are as follows:

An Agilent7890B gas chromatograph is used to analyze products other than glycolic acid and unreacted raw materials, where an FID detector is connected to a DB-FFAP capillary column and a TCD detector is connected to a Porapak Q packed column. A liquid chromatograph is used to analyze glycolic acid, where a separation column is a Cis column and a detector is an ultraviolet (UV) detector.

In an embodiment of the present application, both a conversion rate and selectivity are calculated based on a mole number of carbon:

methyl methoxyacetate conversion rate=[(mole number of carbon in fed methyl methoxyacetate)−(mole number of carbon in discharged methyl methoxyacetate)]÷(mole number of carbon in fed methyl methoxyacetate)×100%;

methoxyacetic acid conversion rate=[(mole number of carbon in fed methoxyacetic acid)−(mole number of carbon in discharged methoxyacetic acid)]÷(mole number of carbon in fed methoxyacetic acid)×100%; and

selectivity for a product=(mole number of carbon in a discharged product)÷(total mole number of carbon in all discharged carbon-containing products)×100%

The present application will be described in detail below with reference to examples, but the present application is not limited to these examples.

Catalyst Performance Test

### Example 1

An acidic H-ZSM-5 molecular sieve with a Si/Al ratio of 20 produced by Zhongke Catalysis New Technology (Dalian) Co., Ltd. was selected, crushed, and sieved to obtain 0.4 mm to 0.8 mm particles; 2 g of the particles was taken and filled into a stainless steel reaction tube with an inner diameter of 8 mm and activated with 50 mL/min nitrogen at 500° C. for 4 h; a reaction was conducted for 24 h under the following conditions: a reaction temperature (T) was 160° C., and a reaction pressure (P) was 0.1 MPa; a molar ratio of methyl methoxyacetate+methoxyacetic acid to water was 1:6, and a molar ratio of methyl methoxyacetate to methoxyacetic acid was 5:1; and a WHSV of methyl methoxyacetate and methoxyacetic acid was 0.6 h⁻¹; and after the reaction was completed, products were analyzed by gas chromatography and liquid chromatography. Reaction results based on a mole number of carbon were shown in Table 1. Negative-ion mass spectrometry results of glycolic acid in liquid chromatography-mass spectrometry analysis of products were shown in the FIGURE.

### Examples 2 to 8

The catalyst, reaction conditions, and reaction results were shown in Table 1. Other operations were the same as in Example 1.

TABLE 1
Catalytic reaction results in Examples 1 to 8

| | | | | | Methyl | | | | |
|---|---|---|---|---|---|---|---|---|---|
| | | | | | methoxy | Methoxyacetic | | | |
| | Acidic | | | | acetate | acid | Glycolic | Methyl | Methanol |
| | molecular | | | | conversion | conversion | acid | glycolate | and DME |
| | sieve | | Si/Al | Reaction | rate | rate | selectivity | selectivity | selectivity |
| Example | catalyst | Manufacturer | ratio | conditions | (%) | (%) | (%) | (%) | (%) |
| 1 | H-ZSM-5 | Zhongke | 20 | T = 160° C.; P = 0.1 | 80.1 | 49.9 | 45.7 | 9.0 | 45.3 |
| | | Catalysis | | MPa; WHSV = 0.6 | | | | | |
| | | New | | h⁻¹; (methyl | | | | | |
| | | Technology | | methoxyacetate + | | | | | |
| | | (Dalian) Co., | | methoxyacetic | | | | | |
| | | Ltd. | | acid):water = 1:6; | | | | | |
| | | | | and (methyl | | | | | |
| | | | | methoxyacetate/ | | | | | |
| | | | | methoxyacetic acid) = | | | | | |
| | | | | 5:1 | | | | | |
| 2 | H-Y | Zhongke | 3 | T = 160° C.; P = 0.1 | 57.9 | 28.7 | 44.3 | 8.4 | 47.3 |
| | | Catalysis | | MPa; WHSV = 0.6 | | | | | |
| | | New | | h⁻¹; (methyl | | | | | |
| | | Technology | | methoxyacetate + | | | | | |
| | | (Dalian) Co., | | methoxyacetic | | | | | |
| | | Ltd. | | acid):water = 1:6; | | | | | |
| | | | | and (methyl | | | | | |
| | | | | methoxyacetate/ | | | | | |
| | | | | methoxyacetic acid) = | | | | | |
| | | | | 5:1 | | | | | |
| 3 | H-ZSM-35 | Catalyst | 50 | T = 160° C.; P = 0.1 | 79.2 | 52.3 | 45.0 | 11.2 | 43.8 |
| | | Plant of | | MPa; WHSV = 0.6 | | | | | |
| | | Nankai | | h⁻¹; (methyl | | | | | |
| | | University | | methoxyacetate + | | | | | |
| | | | | methoxyacetic | | | | | |
| | | | | acid):water = 1:6; | | | | | |
| | | | | and (methyl | | | | | |
| | | | | methoxyacetate/ | | | | | |
| | | | | methoxyacetic acid) = | | | | | |
| | | | | 5:1 | | | | | |
| 4 | H-β | Zhongke | 500 | T = 160° C.; P = 0.1 | 56.2 | 35.9 | 27.7 | 36.0 | 36.3 |
| | | Catalysis | | MPa; WHSV = 0.6 | | | | | |
| | | New | | h⁻¹; (methyl | | | | | |
| | | Technology | | methoxyacetate + | | | | | |
| | | (Dalian) Co., | | methoxyacetic | | | | | |
| | | Ltd. | | acid):water = 1:6; | | | | | |
| | | | | and (methyl | | | | | |
| | | | | methoxyacetate/ | | | | | |
| | | | | methoxyacetic acid) = | | | | | |
| | | | | 5:1 | | | | | |
| 5 | H- | Yanchang | 10 | T = 160° C.; P = 0.1 | 55.5 | 29.3 | 35.7 | 24 | 40.3 |
| | Mordenite | Zhongke | | MPa; WHSV = 0.6 | | | | | |
| | | (Dalian) | | h⁻¹; (methyl | | | | | |
| | | Energy | | methoxyacetate + | | | | | |
| | | Technology | | methoxyacetic | | | | | |
| | | Co., Ltd. | | acid):water = 1:6; | | | | | |
| | | | | and (methyl | | | | | |
| | | | | methoxyacetate/ | | | | | |
| | | | | methoxyacetic acid) = | | | | | |
| | | | | 5:1 | | | | | |
| 6 | H-MCM- | Yanchang | 100 | T = 160° C.; P = 0.1 | 54.3 | 34.2 | 33.7 | 27.0 | 39.3 |
| | 22 | Zhongke | | MPa; WHSV = 0.6 | | | | | |
| | | (Dalian) | | h⁻¹; (methyl | | | | | |
| | | Energy | | methoxyacetate + | | | | | |
| | | Technology | | methoxyacetic | | | | | |
| | | Co., Ltd. | | acid):water = 1:6; | | | | | |
| | | | | and (methyl | | | | | |
| | | | | methoxyacetate/ | | | | | |
| | | | | methoxyacetic acid) = | | | | | |
| | | | | 5:1 | | | | | |
| 7 | H-ZSM-5 | Zhongke | 20 | T = 260° C.; P = 10 | 51.0 | 49.7 | 40.3 | 17.4 | 42.3 |
| | | Catalysis | | MPa; WHSV = 3.0 | | | | | |
| | | New | | h⁻¹; (methyl | | | | | |
| | | Technology | | methoxyacetate + | | | | | |
| | | (Dalian) Co. | | methoxyacetic | | | | | |
| | | Ltd. | | acid):water = 1:20; | | | | | |
| | | | | (methyl | | | | | |
| | | | | methoxyacetate/ | | | | | |
| | | | | methoxyacetic acid) = | | | | | |
| | | | | 4:1; and nitrogen = | | | | | |
| | | | | 100 mL min⁻¹ | | | | | |
| 8 | H-ZSM-5 | Zhongke | 20 | T = 60° C.; P = 0.3 | 24.6 | 31.2 | 42.3 | 16.2 | 41.5 |
| | | Catalysis | | MPa; WHSV = 0.1 | | | | | |
| | | New | | h⁻¹; (methyl | | | | | |
| | | Technology | | methoxyacetate + | | | | | |
| | | (Dalian) Co., | | methoxyacetic | | | | | |
| | | Ltd. | | acid):water = 1:2; | | | | | |
| | | | | and (methyl | | | | | |
| | | | | methoxyacetate/ | | | | | |
| | | | | methoxyacetic acid) = | | | | | |
| | | | | 4:1 | | | | | |

It can be seen from Table 1 that the acidic molecular sieve exhibits excellent catalytic performance in hydrolysis of methyl methoxyacetate and methoxyacetic acid to produce glycolic acid and methyl glycolate, and leads to high selectivity for a target product.

### Example 9

Reaction results of different reaction times in Example 9 were shown in Table 2.

TABLE 2
Catalytic reaction results in Example 9

| | Methyl me- | Methoxyace- | Glycolic | Methyl |
|---|---|---|---|---|
| | thoxyacetate | tic acid | acid | glycolate |
| Reaction | conversion | conversion | selectiv- | selectiv- |
| time (h) | rate (%) | rate (%) | ity (%) | ity (%) |
| 24 | 80.1 | 49.9 | 45.7 | 9.0 |
| 100 | 80.2 | 49.8 | 45.6 | 9.2 |
| 500 | 81.1 | 46.3 | 45.8 | 9.1 |
| 1000 | 78.3 | 47.1 | 45.1 | 8.7 |
| 2000 | 73.3 | 47.1 | 44.8 | 8.2 |
| 4000 | 73.6 | 45.1 | 44.9 | 8.5 |
| 8000 | 73.2 | 44.7 | 44.1 | 9.2 |

It can be seen from Table 2 that the acidic molecular sieve catalyst, especially the H-ZSM-5 molecular sieve catalyst, leads to a high raw material conversion rate during the hydrolysis, and has a long life.

### Example 10

A DB757 strongly-acidic sulfonic acid group-containing exchange resin with an exchange degree of 3.2 mmol/g commercially purchased from Dandong Mingzhu Special Resin Co., Ltd. was used instead of the catalyst in Example 1 and activated with 50 mL/min nitrogen at 100° C. for 4 h, and other conditions and operations were the same as in Example 1. Reaction results were shown in Table 3.

### Example 11

γ-alumina with an ammonia adsorption capacity of 0.29 mmol/g commercially purchased from Beijing Yanxin Technology Development Co., Ltd was used instead of the catalyst in Example 10, and other conditions and operations were the same as in Example 10. Reaction results were shown in Table 3.

### Example 12

A 202 FC strongly-alkaline quaternary ammonium group-containing exchange resin with an exchange degree of 3.5 mmol/g commercially purchased from Dandong Mingzhu Special Resin Co., Ltd. was used instead of the catalyst in Example 10, and other conditions and operations were the same as in Example 10. Reaction results were shown in Table 3.

### Example 13

Hydrotalcite with a composition of [Mg_{0.8}Al_{0.2}(OH)₂]^{0.2+}[CO₃²⁻]_{0.1}·2H₂O was used instead of the catalyst in Example 10, and other conditions and operations were the same as in Example 10. Reaction results were shown in Table 3.

### Example 14

Hydroxyapatite with a composition of Ca₁₀(PO₄)₆ (OH)₂ was used instead of the catalyst in Example 10, and other conditions and operations were the same as in Example 10. Reaction results were shown in Table 3.

TABLE 3
Catalytic reaction results in Example 10 to 14

| | | Methyl me- | Methoxyace- | Glycolic | Methyl |
|---|---|---|---|---|---|
| | | thoxyacetate | tic acid | acid | glycolate |
| | | conversion | conversion | selectiv- | selectiv- |
| Example | Catalyst | rate (%) | rate (%) | ity (%) | ity (%) |
| 10 | Strongly-acidic | 55.3 | 31.2 | 33.7 | 26.3 |
| | sulfonic acid | | | | |
| | group-containing | | | | |
| | exchange resin | | | | |
| 11 | γ-alumina | 25.3 | 25.2 | 31.6 | 24.2 |
| 12 | Strongly-alkaline | 27.3 | 28.2 | 33.6 | 26.2 |
| | quaternary | | | | |
| | ammonium group- | | | | |
| | containing | | | | |
| | exchange resin | | | | |
| 13 | Hydrotalcite | 50.3 | 27.2 | 36.5 | 21.3 |
| 14 | Hydroxyapatite | 32.5 | 30.2 | 30.2 | 30.5 |

It can be seen from Table 3 that the solid acid and base catalysts such as a strongly-acidic resin, γ-alumina, an alkaline resin, hydrotalcite, and hydroxyapatite can also catalyze hydrolysis of methyl methoxyacetate and methoxyacetic acid to produce glycolic acid and methyl glycolate.

### Example 15

86.7 g of methyl methoxyacetate, 15 g of methoxyacetic acid, and 108 g of water were added to a reactor, and 10 mL of a 0.1 mol/L sulfuric acid aqueous solution was added as a catalyst; a reaction was conducted for 24 h under the following conditions: reaction temperature: 160° C., reaction pressure: 0.2 MPa, and stirring speed: 300 rpm; and after the reaction was completed, reaction results were shown in Table 4.

TABLE 4
Catalytic reaction results in Example 15

| | | Methyl me- | Methoxyace- | Glycolic | Methyl |
|---|---|---|---|---|---|
| | | thoxyacetate | tic acid | acid | glycolate |
| | | conversion | conversion | selectiv- | selectiv- |
| Example | Catalyst | rate (%) | rate (%) | ity (%) | ity (%) |
| 15 | Sulfuric acid | 65.3 | 42.2 | 40.8 | 27.9 |
| | aqueous solution | | | | |

It can be seen from Table 4 that the liquid acid can also catalyze hydrolysis of methyl methoxyacetate and methoxyacetic acid to produce glycolic acid and methyl glycolate.

### Example 16

A hydrolysis reaction of methyl methoxyacetate and methoxyacetic acid was tested by a batch catalytic distillation method. A distillation column was a glass column with a diameter of 30 mm, and the distillation column body was filled with an inert annular packing material of 3.0 mm×3.0 mm, with a packing height of 2.0 m. The distillation column was heated by a heating jacket, and a condenser at a top of the column had a temperature of −15° C.

86.7 g of methyl methoxyacetate, 15 g of methoxyacetic acid, and 108 g of water were added to a reactor, and 10 g of the acidic H-ZSM-5 molecular sieve with a Si/A1 ratio of 20 in Example 1 was added as a catalyst; a reaction was conducted for 10 h under the following conditions: reaction temperature: 150° C., reaction pressure: 0.1 MPa, magneton stirring speed: 500 rpm, and reflux ratio: 2; and after the reaction was completed, conversion rates of methyl methoxyacetate and methoxyacetic acid both were about 100%, the selectivity of glycolic acid was 43.5%, and the selectivity of methyl glycolate was 13.0%.

### Example 17

The acidic H-ZSM-5 molecular sieve with a Si/A1 ratio of 20 in Example 1 was molded with alumina or silicon oxide into a strip, and after the molding, a content of the alumina or silicon oxide in the molded catalyst was 20 wt %. Other conditions and operations remained unchanged. Reaction results were shown in Table 5.

TABLE 5
Catalytic reaction results in Examples 1 and 17

| | | Methyl me- | Methoxyace- | Glycolic | Methyl |
|---|---|---|---|---|---|
| | | thoxyacetate | tic acid | acid | glycolate |
| Exam- | Forming | conversion | conversion | selectiv- | selectiv- |
| ple | agent | rate (%) | rate (%) | ity (%) | ity (%) |
| 1 | None | 80.1 | 49.9 | 45.7 | 9.0 |
| 17 | Alumina | 75.3 | 40.2 | 44.2 | 10.1 |
| 17 | Silicon | 75.6 | 41.8 | 44.9 | 10.3 |
| | oxide | | | | |

It can be seen from Table 5 that the catalytic activity of the acidic molecular sieve catalyst remains basically unchanged after the molding with alumina or silicon oxide.

### Example 18

A catalyst obtained after 8,000 h of the reaction in Example 9 was treated with 500 mL min⁻¹ of a mixed gas of oxygen and nitrogen in a molar ratio of 5/95 at 600° C. for 4 h, and then used in the reaction in Example 9. Reaction results were shown in Table 6.

TABLE 6
Catalytic reaction results in Example 18

| | Methyl me- | Methoxyace- | Glycolic | Methyl |
|---|---|---|---|---|
| | thoxyacetate | tic acid | acid | glycolate |
| Reaction | conversion | conversion | selectiv- | selectiv- |
| time (h) | rate (%) | rate (%) | ity (%) | ity (%) |
| 24 | 80.3 | 50.2 | 44.7 | 9.7 |
| 100 | 80.0 | 49.7 | 44.8 | 9.6 |
| 500 | 79.5 | 48.8 | 45.3 | 8.9 |

It can be seen from Table 6 that, after being calcined and regenerated with the oxygen/nitrogen mixed gas, the reacted catalyst can be basically restored to reaction performance of a fresh catalyst.

The above examples are merely few examples of the present application, and do not limit the present application in any form. Although the present application is disclosed as above with preferred examples, the present application is not limited thereto. Some changes or modifications made by any technical personnel familiar with the profession using the technical content disclosed above without departing from the scope of the technical solutions of the present application are equivalent to equivalent implementation cases and fall within the scope of the technical solutions.

What is claimed is:

1. A method for preparing glycolic acid and methyl glycolate through a hydrolysis of methyl methoxyacetate and methoxyacetic acid, comprising: allowing raw materials comprising the methyl methoxyacetate, the methoxyacetic acid, and water to a contact and a reaction with a catalyst to produce the glycolic acid and the methyl glycolate,
wherein the catalyst is one selected from the group consisting of a solid acid catalyst, a liquid acid catalyst, a solid base catalyst, and a liquid base catalyst.

2. The method according to claim 1, wherein the solid acid catalyst is at least one selected from the group consisting of an acidic molecular sieve catalyst, an acidic resin catalyst, and an acidic alumina catalyst; and
the acidic molecular sieve catalyst comprises an acidic molecular sieve.

3. The method according to claim 2, wherein the acidic molecular sieve is at least one selected from the group consisting of an acidic MFI-structured molecular sieve, an acidic FAU-structured molecular sieve, an acidic FER-structured molecular sieve, an acidic BEA-structured molecular sieve, an acidic MOR-structured molecular sieve, and an acidic MWW-structured molecular sieve, or
the acidic molecular sieve is at least one selected from the group consisting of an acidic ZSM-5 molecular sieve, an acidic Y molecular sieve, an acidic ZSM-35 molecular sieve, an acidic β molecular sieve, an acidic MOR molecular sieve, and an acidic MCM-22 molecular sieve; or
the acidic molecular sieve is at least one selected from the group consisting of a hydrogen-type ZSM-5 molecular sieve, a hydrogen-type Y molecular sieve, a hydrogen-type ZSM-35 molecular sieve, a hydrogen-type β molecular sieve, a hydrogen-type MOR molecular sieve, and a hydrogen-type MCM-22 molecular sieve.

4. (canceled)

5. (canceled)

6. The method according to claim 2, wherein a Si/Al atom ratio of the acidic molecular sieve is 3 to 500; and/or
a content of the acidic molecular sieve in the acidic molecular sieve catalyst is 50 wt % to 100 wt %.

7. (canceled)

8. The method according to claim 2, wherein the acidic molecular sieve catalyst further comprises a forming agent;
the forming agent is an oxide; and
the oxide is one selected from the group consisting of alumina and silicon oxide;
preferably, a content of the forming agent in the acidic molecular sieve catalyst is m, and 0<m≤50 wt %.

9. (canceled)

10. The method according to claim 2, wherein the acidic molecular sieve catalyst is a fresh acidic molecular sieve catalyst and/or a regenerated acidic molecular sieve catalyst; and
the fresh acidic molecular sieve catalyst is an unused acidic molecular sieve catalyst.

11. The method according to claim 10, wherein a regeneration method of the acidic molecular sieve catalyst comprises:
treating an inactivated acidic molecular sieve catalyst with an oxygen-containing regeneration gas at 400° C. to 800° C. for 0.5 h to 24 h to obtain the regenerated acidic molecular sieve catalyst,
wherein a volume fraction of oxygen in the oxygen-containing regeneration gas is 0.5% to 50%.

12. The method according to claim 2, wherein the acidic resin catalyst is a strongly-acidic cation exchange resin.

13. The method according to claim 12, wherein a skeleton structure in the strongly-acidic cation exchange resin is a copolymer of styrene and divinylbenzene (DVB); and
an acidic group in the strongly-acidic cation exchange resin is a sulfonic acid group.

14. The method according to claim 2, wherein the acidic alumina catalyst is γ-alumina.

15. The method according to claim 1, wherein the liquid acid catalyst is an acidic liquid.

16. The method according to claim 15, wherein the liquid acid catalyst is at least one selected from the group consisting of sulfuric acid, hydrochloric acid, nitric acid, and phosphoric acid;
preferably, a concentration of H⁺ in the liquid acid catalyst is 0.01 mol/L to 10 mol/L.

17. (canceled)

18. The method according to claim 1, wherein the solid base catalyst is at least one selected from the group consisting of hydrotalcite, an anion exchange resin, and hydroxyapatite.

19. The method according to claim 1, wherein the liquid base catalyst is an alkaline liquid;
preferably, the liquid base catalyst is one selected from the group consisting of a sodium hydroxide aqueous solution, a potassium hydroxide aqueous solution, a calcium hydroxide aqueous solution, and a magnesium hydroxide aqueous solution; and
a concentration of OH⁻ in the liquid base catalyst is 0.01 mol/L to 10 mol/L.

20. (canceled)

21. (canceled)

22. The method according to claim 1, wherein conditions of the reaction are as follows:
a reaction temperature is 60° C. to 260° C.;
a reaction pressure is 0.1 MPa to 10 MPa; and
in the raw materials, a ratio of a total mole number of the methyl methoxyacetate and the methoxyacetic acid to a mole number of the water is 1:2 to 1:20;
preferably,
the reaction temperature is 130° C. to 200° C.;
the reaction pressure is 0.1 MPa to 0.3 MPa; and
in the raw materials, the ratio of the total mole number of the methyl methoxyacetate and the methoxyacetic acid to the mole number of the water is 1:3 to 1:8 and
a molar ratio of the methyl methoxyacetate to the methoxyacetic acid is 4:1 to 9:1.

23. (canceled)

24. The method according to claim 1, wherein the reaction is conducted in a reactor; and
the reactor is one selected from the group consisting of a fixed-bed reactor, a tank reactor, and a catalytic distillation reactor.

25. The method according to claim 24, wherein the reactor comprises a single fixed-bed reactor, or a plurality of fixed-bed reactors connected in series and/or parallel; or
the reactor comprises a single tank reactor, or a plurality of tank reactors connected in series and/or parallel; or
the reactor comprises a single catalytic distillation reactor, or a plurality of catalytic distillation reactors connected in series and/or parallel.

26. The method according to claim 24, wherein when the fixed-bed reactor is adopted,
a weight hourly space velocity (WHSV) of the methyl methoxyacetate and the methoxyacetic acid in the raw materials is 0.1 h⁻¹ to 3 h⁻¹;
when the tank reactor is adopted, a stirring speed is 250 rpm to 350 rpm; and a reaction time is 1 d to 3 d; and
when the catalytic distillation reactor is adopted, a reaction time is 8 h to 15 h; a stirring speed is 350 rpm to 650 rpm; and a reflux ratio is 1 to 3.

27. (canceled)

28. (canceled)

29. The method according to claim 1, wherein the methyl methoxyacetate in the raw materials comprises a freshly-added raw material and/or unreacted methyl methoxyacetate left after a product is separated; and/or,
the methoxyacetic acid in the raw materials comprises a freshly-added raw material and/or unreacted methoxyacetic acid left after a product is separated; and/or,
the water in the raw materials comprises a freshly-added raw material and/or unreacted water left after a product is separated.

30. The method according to claim 1, wherein the reaction is conducted in an inactive atmosphere; and
the inactive atmosphere comprises one selected from the group consisting of nitrogen and an inert gas.
